(19) United States
(12) Patent Application Publication
(10) Pub. No.: US 2004/0023952 A1
(43) Pub. Date: Feb. 5, 2004

(54) Enhanced brain function by gaba-ergic stimulation

(76) Inventor: Audie G. Leventhal, Salt Lake City, UT (US)

Correspondence Address: KLARQUIST SPARKMAN, LLP, 121 SW SALMON STREET, SUITE 1600, PORTLAND, OR, 97204, US

(21) Appl. No.: 10/311,821

(22) PCT Filed: Jun. 20, 2001
(86) PCT No.: PCT/US01/19719

(51) Int. Cl. A61K 31/5513; A61K 31/515; A61K 31/19; A61K 31/195

(52) U.S. Cl. 514/221; 514/270; 514/561; 514/557

(57) ABSTRACT

Methods are disclosed for improving age-related decreases in cortical function by increasing the activity of inhibitory pathways, such as GABA-ergic pathways, in the central nervous system. In particular examples, subjects with age-related decreases in cortical function are treated by administering therapeutically effective amounts of a GABA-ergic agonist. The disclosed methods also enable screening for drugs that inhibit an age-related decline in cortical function, for example by exposing a subject to a test agent, and measuring an increase in GABA-ergic cortical inhibitory activity.

### FIELD OF THE INVENTION

This invention concerns treatments for improving age-related cortical function of a subject.

### BACKGROUND

Cognition is the ability of a subject to use information about and from the environment in an adaptive way. Unfortunately, cognitive and other cortical functions (such as auditory discrimination, somatosensory function, motor function, and language abilities) often decline in aging subjects. This decline is a common cause of incapacity, morbidity and even death in elderly animals and humans. These problems are expected to become more widespread as life span increases, and more individuals live into senescence. One of the great medical and social challenges of the coming decades is to develop approaches to deal with this often incapacitating problem.

Gamma-aminobutyric acid (GABA) is regarded as one of the major inhibitory amino acid transmitters in the mammalian brain. Widely (although unequally) distributed through the mammalian brain, GABA is believed to be a transmitter at approximately 30% of the synapses in the brain. GABA mediates many of its actions through a complex of proteins (GABA receptors) localized both on cell bodies and nerve endings. Postsynaptic responses to GABA are mediated through alterations in chloride conductance that generally, although not invariably, lead to hyperpolarization of the cell. Drugs that interact at the GABAa receptor can possess a spectrum of pharmacological activities depending on their abilities to modify the action of GABA.

One example of a GABA agonist is a benzodiazepine receptor agonist, such as diazepam or chlordiazepoxide. These 1,4-benzodiazepines, such as diazepam are among the most widely used drugs in the world as anxiolytics, muscle relaxants, and anticonvulsants. A number of these compounds are extremely potent drugs; such potency indicates a site of action with a high affinity and specificity for individual receptors. Early electrophysiological studies indicated that a major action of benzodiazepines was enhancement of GABAergic inhibition of the central nervous system. Compounds which have activity opposite to benzodiazepines are called inverse agonists, and compounds blocking both types of activity have been termed antagonists.

The GABA receptor subunits are categorized as alpha, beta, gamma, delta and epsilon, and they provide a molecular explanation for the GABA receptor heterogeneity, and distinctive regional pharmacology. The gamma subunit appears to enable drugs like benzodiazepines to modify the GABA responses. Depending on the mode of interaction, these compounds are capable of producing a spectrum of activities, such as sedation, anxiolysis, anticonvulsant activity, or wakefulness, seizures, or anxiety. It is generally accepted that GABA agonists provide cortical inhibition which impairs cognitive and other cortical activities, and are to be avoided in situations wherein optimal higher cortical functions (such as thinking and visual perceptual) are required. GABA inverse agonists, which block the cortical inhibitory action mediated by GABA receptors, have been proposed as treatments for cognitive disorders, such as Alzheimer's disease (see e.g. WO 99/06401, which is incorporated by reference).

Like cognition, human visual function declines with age. This decline has usually been attributed to abnormalities in the optical properties of the eye, such as cataracts (opacities in the crystalline lens of the eye) or retinal degeneration (for example of the type that is seen in age related macular degeneration). Hence visual research and care for the elderly primarily involves addressing these problems, for example by extraction of cataracts and treatment of choroidal neovascularization that precedes macular degeneration.

### SUMMARY OF THE DISCLOSURE

It has now surprisingly been found that at least a portion of visual dysfunction in elderly individuals is the result of degenerative changes in cortical function, such as the central visual pathways. This degenerative change is functionally manifested by a decrease in the activity of central inhibitory pathways, and particularly by the GABA-ergic inhibitory pathways. The result is that the peak response and spontaneous activity of cerebral cortical cells is abnormally high in older individuals, and that there is a significant loss of inhibitory activity that leads to degradation of visual, auditory, somatosensory, motor and/or language functions of the brain. For example, in the visual pathways, orientation and direction selectivity decreases in aging subjects, with a decreased signal to noise ratio. These changes are demonstrated, for example, in primary visual cortex (striate cortex or VI) in very old macaque monkeys using single-neuron in vivo electrophysiology. Decreased selectivity of cells in old animals was accompanied by increased responsiveness to all orientations and directions, as well as an increase in spontaneous activity. The decreased selectivities and increased excitability of cells in old animals are believed to be part of a more wide-spread age-related degeneration of intracortical inhibition.

Certain disclosed embodiments include treating a subject having age-related decreases in cortical function by administering to the subject a therapeutically effective amount of a GABA-ergic agonist. In particular examples, the age-related decrease in cortical function is a decrease in cognitive function or visual function (such as a decrease in orientation and direction selectivity). In other examples, the GABA-ergic agonist is a GABA-A, GABA-B, or GABA-C receptor agonist, such as a benzodiazepine receptor agonist, and in particular a member of the class of drugs known as benzodiazepines. Other examples of the GABA-ergic agonist include GABA, muscimol, baclofen, CaCa, valproic acid, a barbiturate, gabapentin, tigabine, or vigabatrin.

In some examples, the method includes determining, prior to treating the subject, whether the subject has an age-related decrease in GABA-ergic activity, such as an age-related decrease in visual orientation and direction selectivity, auditory frequency discrimination and/or sound localization, somatosensory function (such as a decrease in an ability to detect quality, intensity or position of sensation), motor function (such as a control of voluntary movements), and/or language ability (such as a decrease in speech comprehension and/or generation, such as sentence formation). The electrophysiological changes in these visual functions can be used as a diagnostic marker for more widespread cortical loss of GABA-ergic activity that can be treated using the methods disclosed herein.

Also disclosed are methods of screening for agents that inhibit age related cortical decline, such as visual and cognitive decline, by determining whether a test agent increases GABA-ergic cortical inhibitory activity. In particular examples, the assay involves administering a test agent, and measuring a change in a neuron in a specific area of the brain that is associated with age-related decline. Among the many specific examples provided in the detailed examples are orientation bias, direction bias, spontaneous activity, or a signal to noise ratio in spontaneous baseline frequencies in selected areas of the cortex, such as the visual cortex, for example V1. GABA-ergic agents that increase orientation bias, direction bias, or the signal to noise ratio, or decrease spontaneous baseline frequencies, are then selected for further testing in cognitive and visual function studies. Alternatively, a decreased spontaneous baseline frequency or an increased signal to noise ratio in many other areas of the brain (such as the auditory, somatosensory and/or language centers) can be used to select for GABA-ergic agents that will improve cortical function by decreasing the spontaneous cortical activity that masks efficient neurotransmission in the aging brain.

### BRIEF DESCRIPTION OF THE FIGURES

FIG. 1 shows photomicrographs of whole-mounted, HRP-labeled retinae and single intracellularly dye-filled ganglion cells. The ganglion cell density within and surrounding the foveal pit appeals qualitatively normal in the old monkey (a) compared to the young monkey (b)(scale bars, 500 μm). The ratio of A (Pα or parasol) and B (pβ or midget) ganglion cells also appears normal in old monkey (c), in which the photomicrograph is taken 4 mm from the center of the foveal pit (scale bar 50 μm). Type B cells have characteristically smaller soma and dendritic fields relative to A cells. Confocal microscope optical sectioning of intracellularly injected, CY-3-reacted cells in the opposite eye of the same old monkey revealed that both A and B cells retain characteristic soma diameter and dendritic field size, ramification and branching pattern. Proportional increase in A cell dendritic field diameter, with increasing retinal eccentricity from 3.9 mm (d) to 7.4 mm (e)(scale bars, 50 μm, arrows indicate axons). Side and bottom panels show the y-z and x-z panes, respectively, illustrating normal dendritic arborization in the inner plexiform layer, above the retinal ganglion cell layer containing the cell bodies.

FIG. 2 is a series of graphs showing orientation and direction biases in young and old macaque V1 cells which were exposed to either drifting sinusoidal gratings or drifting luminance bar stimuli. Orientation biases of 0.1, 0.3 and 0.5 correspond to maximum-to-minimum response ratios of 1.5:1, 3:7:1 and 10.8:1. respectively. An orientation bias of 0.1 or greater indicates significance at the p>0.005 level (Rayleigh test). The data from cells from individual young (n=187) and old monkeys(n=254) are shown in scatterplots (a, b). The percentage of cells with any given orientation bias value are shown in cumulative distribution plots (c, d) where solid black and gray lines represent the combined data of old and young monkeys. Pearson product moment correlations between OB and D8 values for young (r=0.46) and old (r=056) monkeys were significant (p>0.05).

FIG. 3 shows tuning curves and corresponding polar plots obtained from four old monkey cells. Responses are shown to drifting luminance bars (a, b) and sinusoidal gratings (c, d) of systematically varied orientation and direction. The responses of two selective and two nonselective cells are provided for comparison. Orientation biases for each plot are 0.307 (a), 0.042 (b), 0.505 (c) and 0.081 (d). Direction biases are 0.065 (a), 0.018 (b), 0.118 (c) and 0.023 (d). The orientations of the driving gratings and bars are orthogonal to the directions indicated. Each point in the polar plots represents the response for the stimulus moving in the indicated direction. One-half the length of the axes intersecting at the corner of each polar plot was made equal to the maximum response for each tuning curve. All other responses were scaled to represent the percent maximum response. Histograms surrounding the polar plots demonstrate the cellular response as a function of time. For (a) and (b), spikes were placed in 100-ms bins and summed for the 5 stimulus sweeps per orientation or direction. For (c) and (d), spikes were placed in 20-ms bins and summed for 18 cycles of the sinusoidal grating.

FIG. 4 is a series of graphs which illustrates the relationship between orientation biases and peak visual evoked response of young monkeys (a, c, e) and old monkeys (b, d, f). FIGS. 4(a) and 4(b) shows the relationship between orientation biases and peak visual evoked response (baseline subtracted) of young (a) and old (b) monkey VI cells to drifting bar stimuli. FIGS. 4(c) and (d) show the relationship between orientation bias and peak FFTI response of different young (c) and old (d) monkey VI cells to drifting sinusoidal gratings. FIGS. 4(e) and (f) show the relationship between peak visual evoked response and baseline activity of young (e) and old (f) monkey VI cells. Cells shown in (a) and (b) are identical to those shown in (e) and (f) respectively. Selective and nonselective cells in both age groups show a wide range of peak amplitudes. Sample sizes (n) and average peak amplitudes (X) in spikes per s are n=111, X=47.8(a); n=101, X=82.6 (b); n=77, X=43.5 (c); n=153, X=87.2 (d); n=109, X=3.5 (e); n=101, X=21.6 (f). Average peak amplitudes were significantly increased in old monkeys for both drifting bar and drifting sinusoidal grading data sets (p<0.05). Cells with peak amplitudes>200 Hz in the drifting sinusoidal grating orientation data set (n=7) were used in all statistical comparisons but were removed from the scatterplots to increase resolution of most cells.

FIGS. 5-9 are a series of bar graphs which illustrate the peak response (in spikes/second) in old monkeys and young monkeys, as well as in old monkeys which are given GABA, muscimol, or bicuculline.

FIGS. 10A-F shows tuning curves and corresponding polar plots for monkeys that received treatment with GABA, a GABA agonist (muscimol) and a GABA antagonist (bicuculline).

### DETAILED DESCRIPTION OF PARTICULAR EXAMPLES

Unless otherwise noted, technical terms are used according to conventional usage. In order to facilitate review of the various embodiments of the disclosed methods, the following explanations of specific terms are provided As used herein and in the appended claims, the singular forms “a”, “an”, and “the” include plural referents unless the context clearly dictates otherwise. Thus, for example, reference to “a cell” includes a plurality of such cells and reference to “the cell” includes reference to one or more cells, and so forth.

An “agent” includes conventional chemical pharmaceutical compounds, as well as polypeptides, peptidomimetics, biological agents, antibodies or other molecules with a desired function.

An “animal” is a living multicellular vertebrate organism, a category which includes, for example, mammals and birds. A “mammal” includes both human and non-human mammals. Similarly, the term “subject” includes both human and veterinary subjects.

“Cortical function” refers to function of the cortex of the brain, as measured either functionally by neurological testing, or electrophysiologically, for example by a decreased signal to noise ratio.

A “GABA-ergic” agent is an agent that exerts a GABA-like effect, and include GABA-agonists and agents that have effects like GABA-agonists.

A “therapeutically effective amount” is a quantity of an agent sufficient to achieve a desired effect in a subject being treated. In one specific, non-limiting example, a therapeutically effective amount of a GABA-ergic agent is the amount necessary to improve cortical functioning, for example as measured by an improvement in cognition, somatosensory, visual or auditory function. When administered to a subject, a dosage will generally be used that will achieve target tissue concentrations (for example, in neurons of the CNS) that has been shown to achieve improvements in cognition using direct neuronal administration (as described in Examples 1-2 and 8).

Studies of visual perception indicate that aged humans show decreased visual acuity, binocular summation, contrast sensitivity, motion sensitivity and wavelength sensitivity.¹⁻⁴ Senescent humans also respond much more slowly in visual tests^{4,5}, and cannot perform as well in shape discrimination tests^{1,6-9}. This decline has often been attributed to intrinsic ocular pathology in the elderly, as opposed to perceptual defects. As disclosed herein, however, this visual decline is at least in part a result of a decline in inhibitory GABA-ergic pathways in the brain, which have widespread effects on sensory, somatosensory, motor and language abilities, and which can be reversed by administering GABA-ergic agents to the subject.

### EXAMPLE 1

### Preparation for Extracellular Single-Neuron Recording Techniques

Extracellular single-neuron recording techniques were used to examine the stimulus selectivities of VI cells in very old rhesus monkeys showing normal optics and retinogeniculate projections. A total of 187 neurons were studied in 4 young rhesus monkeys (Maraca mulatta), and 254 neurons in 4 old rhesus monkeys. Subjects for physiological experiments were abbreviated as OM1-4 (for old monkey 1-4) and YM1-4 (for young monkey 1-4). Retinal data were obtained from one additional young macaque (YM5, Macaca fascicularis). Multiple ophthalmological exams were conducted to ensure optical and retinal health of all subjects prior to testing.

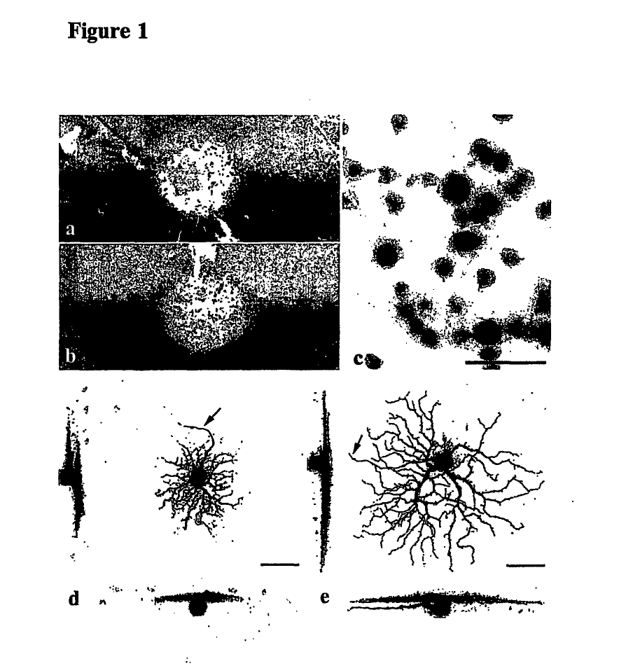

Subjects for this study were four young adult (7-9 year old) and four very old (28-30 year old) female rhesus monkeys (Macaca mullata). A life-span analysis of rhesus macaques housed at this center found that only 25% reached the age of 25, and only 6% reached the age of 30 or older. Thus, the 28-30 year old monkeys were considered old, whereas the 7-9 year old monkeys were at an age considered sexually mature. Retinal control data for FIG. 1 are provided from one additional young female Macaca facsicularis, which was used in previous studies. Onset latency data from YM4 are published. Monkeys were examined ophthalmoscopically, and no apparent optical or retinal problems were detected that would impair visual function. Retinal blood vessels, lens clarity and the maculae all appeared normal. All cells studied had receptive fields between 2 and 5 degrees from the fovea. Monkeys were prepared for electrophysiological recording using standard techniques consistent with Society for Neuroscience and National Institute of Health guidelines.

Subjects were sedated with Ketamine HC1 (Ketalar, Parke-Davis, Morris Plains, N.J.) and then anesthetized with halothane (5%; Halocarton Laboratories, River Edge, N.J.) in a 70:30 mixture of NO₂:O₂. Intravenous and tracheal cannulae were inserted, the animals were placed in a stereotaxic apparatus, and pressure points and incisions were infiltrated with a long-acting anesthetic (2% lidocaine HCI, Copley Pharmaccuticak, Canton, Mass.). A mixture of D-tubocurarine (0.4 mg per kg per hour) and gallamine triethiodide (7 mg per kg per hour) was infused intravenously to induce and maintain paralysis. Animals were ventilated, and anesthesia was maintained with a mixture of nitrous oxide (75%), oxygen (25%) and halothane (0.25-1.0%) as needed.

The level of anesthesia was adjusted so that vital signs were comparable in old and young animals. A small burr hole was made above the striate cortex (V1), and filled with a 4% solution of agar in saline and sealed with wax. The eyes were protected from desiccation with contact lenses; spectacle lenses and artificial pupils were used when needed to focus the eyes on a tangent screen positioned 228 cm from the retina. The locations of the optic discs and foveae were determined repeatedly during the course of each recording session. No visible deterioration in optics occurred during the experimental period (2-5 days).

Extracellular action potentials of isolated cortical cells were recorded with microelectrodes having impedances of 3-5 MΩ. The electrode was advanced using a hydraulic microdrive (David Kopf instruments, Tojunga, Calif.) to precisely position it. The position of the electrode was confirmed by determining the receptive filed positions of the cortical cells at the recording site. A1 V1 cells studied had receptive fields between about 3 and 7 degrees from the fovea.

### EXAMPLE 2

### Visual Stimulation for Single Neuron Recording

After placing the microelectrode as in Example 1, visual stimuli were measured using a Tektronix (Beaverton, Oreg.) 608 display driven by a Picasso (Inning Cambridge, Mass.) image synthesizer and a texture/motion generator (Innisfree). The Picasso and texture/motion generator were computer controlled in conjunction with a hardware and software package from Cambridge Electronics Design (Cambridge, England). The center of the display screen was 171 cm from the animal's retina. Computer-generated stimuli were presented monocularly to the dominant eye in all cases but three, in which cells showed clear binocular summation and consequently drifting stimuli were presented binocularly.

The physiological orientation biases and direction biases of cortical cell were studied quantitatively. The orientation of each drifting stimulus presented was orthogonal to its direction of motion. (The orientation is 90° less than the direction.) Five to twenty presentations of moving bars, spots, or sinusoidal gratings at each of 24-36 randomly generated orientations or directions from 0° to 360° were used to compile the tuning curves for the cells studied. The responses of the cells were studied at a variety of spatial frequencies (cycles per degree) when gratings were used. Each cell's optimal size and temporal frequency/velocity was chosen for the drifting stimulus. In general, each cell provided quantitative orientation bias (OB) and direction bias (DB) values in response to 2-6 different stimulus sets, and some in response to as many as 12. The maximum OB obtained for each cell was included in the data set, along with the maximal DB obtained from either the same stimulus presentation or from a different stimulus set, where the preferred direction was similar but the orientation bias was sub-maximal. All of the orientation and direction biases were taken from either drifting bar stimulation (OBs, 212 of 441 neurons; DBs, 200 of 441) or drifting sinusoidal grating stimulation (OBs 229 neurons; DBs, 241).

The luminance of the stimuli used was 837 cd per m² for white spots and bars, and 0.91 cd per m² for black spots and bars. The contrast for bar and spot stimuli was defined as the ratio of the luminance of the spot or bar to its background. The contrast for sinusoidal gratings was defined as the ratio of the luminance of the center of the light and dark cycles of the gratings. In both cases the contrast was kept at 80% [(8.37-0.91 cd per m²)/(8.37+0.91 cd perm²)].

### EXAMPLE 3

### Analysis of Orientation and Direction Selectivities

The responses of the cells to the drifting visual stimuli presented were stored electronically for later analysis. The responses to the sinusoidal gratings were fast Fourier transformed (FFT) and defined as the peak-to-peak value of the fundamental Fourier component (F1) of the poststimulus time histogram integrated over a time equaling the stimulus modulation period (FFTI spikes per s). For stimuli other than gratings, the responses were defined as the peak response (in Hz) of the post-stimulus time histogram. As each drifting bar was presented, baseline values were obtained during a 0.5-0.67 second blank stimulus period. All baseline values below 1 spike per second were set equal to 1 spike per s for peak-to-baseline analyses. This modification reduced skewing of the data and provided a more conservative estimation of aging differences because many young monkey cells would have peak-to-baseline ratios well above 100 before modification.

Orientation and direction selectivity were calculated for each cell using the statistical methods disclosed in Leventhal et al., J. Neurosci. 15:1808-1818, 1995. Briefly, the responses of each call to the different stimulus orientations and directions were stored as a series of vectors. The vectors were added and divided by the sum of the absolute values of the vectors. The angle of the resultant vector gave the preferred orientation and direction of the cell. The length of the resultant vector, termed the orientation or direction bias, provided a quantitative measure of the orientation or direction sensitivity of the cell. A bias of 0.1 is significant at the p<0.005 level (Raleigh test) and orientation biases of 0.1, 0.3 and 0.5 correspond to maximum to minimum response ratios of 1.5:1, 3.7:1 and 10.8:1, respectively. Hence number higher than 0.1 indicate better selectivity bias.

Statistical comparisons between young and old monkey data were carried out in two ways. The first approach compared the entire data set of each old monkey to that of each young monkey using one-way ANOVAs, t-tests, Kruskal-Wallis one-way ANOVAs and/or Mann-Whitney rank sum tests, as appropriate. The second approach reduced the data set of each monkey to the average score, and compared young monkeys versus old monkeys using these single data points (t-test or Mann-Whitney rank sum test). The results of these two approaches were virtually identical in all cases.

### EXAMPLE 4

### Retinal Histology, Intracellular Injection, Immunohistochemistry and Confocal Microscopy

Eyes were enucleated and retinae were either reacted for horseradish peroxidase (as described in Leventhal et al., Science 213:1139-1142, 1981) or prepared for intracellular injection. In the latter case, retinae were transferred to an injection chamber and superfused with oxygenated Ames medium (Sigma) at a flow rate of about 4 ml per minute at room temperature. Cells were visualized with 0.5% acridine orange (Sigma). Under visual control, cells were penetrated electrically or mechanically with an injection electrode containing 4% Lucifer yellow (CH dilithium salt, Sigma) and 3% biocytin (Molecular Probes, Eugene, Oreg.). A small biphasic current pulse (up to 2 nA hyperpolarizng and 0.5 nA depolarizing for 1-3 min) was applied to inject the dye. After completion of injections, retinae were fixed for about 12 hours in 4% paraformaldehyde in 0.1 M phosphate buffer (pH 7.4) at 4° C. Subsequent steps are as described in Pu and Berson, J. Neurosci. Methods 41:45-49, 1992. However, the final step was changed by replacing avidinbiotin-HRP immunoreaction with avidinbiotin-CY-3 immunoreaction (Jackson Laboratory, Philadelphia, Pa.) to permit confocal microscopy. CY3-labeled ganglion cells were scanned with a Zeiss laser scanning confocal microscope (LSM510) as described in Pu, J. Comp. Neurol. 414:267-274, 1999.

At the conclusion of each experiment, the animal was deeply anesthetized and perfused through the heart with 700 ml of lactated Ringer's solution containing 0.1% heparin, followed by 1000 ml of 1% paraformaldehyde and 2.5% glutaraldehyde in 0.1 M phosphate buffer at pH 7.4, followed by 600 ml of lactated Ringer's solution containing 5% dextrose. Brains were removed, and the locations of the electrode tracks were determined as in Leventhal et al., J. Neurosci., 1995.

### EXAMPLE 5

### Ganglion Cell Density Normal

Injections of horseradish peroxidase (HRP) into the dorsal lateral geniculate nucleus (LGNd) of one hemisphere provided intense ganglion cell labeling along the nasotemporal division in OM4 (FIG. 1a) and YM5 (FIG. 1b). In OM4, qualitatively normal ganglion cell density was found (both within and surrounding the fovea), and the proportion of A (Pα or parasol) and B (Pβ or midget) cells (FIG. 1c) and macular anatomy were normal. Optical sectioning of intracellularly injected cells in the opposite eye of the same animal revealed that both A and B cells retained characteristic soma diameter and dendritic field size, structure and branching patterns (FIGS. 1d and 1e). Soma diameter and dendritic field size varied with retinal eccentricity, as expected. These results confirmed the ophthalmological examinations, and indicated that extreme age does not have an apparent affect on retinal morphology or ganglion cell projections in otherwise healthy monkeys.

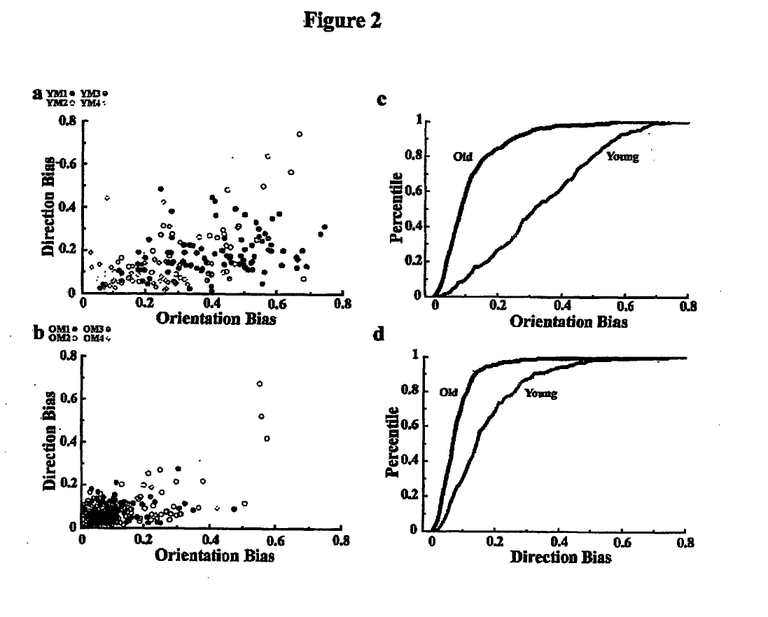

Orientation and direction biases (OB and DB, respectively) were calculated for the 187 young (YM1-4) and 254 old (OM1-4) macaque V1 cell using drifting bar and sinusoidal grating stimuli. The percentage of V1 neurons showing significant OB (≧0.1) was smaller for old macaques (42%; 107 of 254) than for young macaques (90%; 169 of 187). Similarly, the percentage of cells that were strongly biased for orientation (OB≧0.2) was lower for old macaques (15%; 39 of 254) than for young macaques (73%; 137 of 187). Median OB values showed significant inter-animal variability for old monkeys (Kruskal-Wallis ANOVA H=14.1, p<0.05) and young monkeys (H=62.4, p<0.01). However, with one exception, the mean (t-test) or median (Mann-Whitney rank sum test) orientation bias for each individual old monkey was significantly less than that for any individual young monkey (FIG. 2, Table 1). There was no significant difference in median OBs for OM1 (0.079) and YM4 (0.156; p>0.05). A separate analysis compared the average OBs for young monkeys versus old monkeys and also showed a significant aging effect, t₆=3.9. p<0.01.

The percentage of neurons in normal young monkey V1 that showed a significant direction bias was smaller than the percentage of orientation-sensitive cells (FIG. 2). As with orientation bias, the percentage of cells showing significant direction bias in the old monkey. (25%; 84 of 254) was less than that for the young monkey (70%; 130 of 187). The percentage of cells showing strong direction selectivity (DB≧0.2) in the old monkey (5%; 12 of 254) was also less than that for the young monkey (31%; 58 of 187). Median DB values showed significant interanimal variability for young monkeys (H=30.4, p<0.01) but not for old monkeys (H=6.9, p=0.08). Thus, the mean or median DB for each individual young monkey was compared with that for the old monkey population (Table 1) and was significantly greater in each case (p<0.01). Also, with one exception, the mean (t-test) or median (Mann-Whitney rank-sum test) direction bias for individual old monkeys was significantly less than that for any individual young monkey (FIG. 2, Table 1). A separate analysis compared the average direction biases for young monkeys versus old monkeys and also showed a significant aging effect (Mann-Whitney test, p<0.05).

TABLE 1
Descriptive Statistics of Orientation and Direction Biases

| | Median | Mean | std. | 75% | Max | n |
|---|---|---|---|---|---|---|
| Orientation bias | | | | | | |
| OMI | 0.079 | 0.145 | 0.135 | 0.2 | 0.475 | 15 |
| OM2 | 0.101 | 0.136 | Q115 | 0.183 | 0.574 | 115 |
| OM3 | 0.085 | Q094 | Q065 | 0.112 | 0.305 | 78 |
| OM4 | 0.056 | 0.086 | 0.085 | 0.105 | 0.423 | 46 |
| YM1 | 0.407 | 0.416 | 0.148 | 0.521 | 0.743 | 56 |
| YM2 | 0394 | 0372 | 0.156 | 0.458 | 0.679 | 35 |
| YM3 | 0.4 | 0386 | 0.169 | 0524 | 0.687 | 44 |
| YM4 | 0.156 | 0.177 | 0.112 | 0.26 | 0.481 | 52 |
| Old | 0.086 | 0.115 | Q1 | 0.142 | 0574 | 254 |
| Young | 0317 | 0.334 | 0.175 | 0.462 | 0.743 | 187 |
| Direction bias | | | | | | |
| OM1 | 0.086 | 0.094 | Q038 | 0.117 | 0.181 | 15 |
| OM2 | 0.069 | 0.088 | Q092 | 0.101 | 0.676 | 11 5 |
| OM3 | 0.066 | 0.076 | 0.048 | Q106 | 0.277 | 78 |
| OM4 | 0.062 | 0.067 | Q054 | 0.084 | 0326 | 46 |
| YMI | Q191 | 0˜06 | 0.i11 | 0.26i | 0.486 | 56 |
| Yb12 | 0.202 | 0.234 | 0.173 | 0.279 | 0.746 | 35 |
| YM3 | 0.133 | 0.138 | 0.064 | 0.177 | 0.282 | 44 |
| YM4 | 0.084 | 0.124 | 0.M9 | 0.144 | 0.641 | 52 |
| Old | 0.067 | 0.081 | o.on | 0.1 | 0.676 | 252 |
| Young | 0.142 | Q173 | 0.126 | 0.2D | 0.746 | 187 |

Multiple two group comparisons of orientation and direction bias data were carried out between individual young and old monkeys via parametric (t-test) or non-parametric (Mann-Whitney rank sum test) tests as appropriate. Because Mann-Whitney tests were carried out on median values, rather than means, both values are included. Standard deviations are based on means. Old and young rows represent data collapsed across subjects.

Even though inter-animal variability existed in both old and young monkey populations, significant aging effects were observed (FIG. 2). However, a number of old monkey neurons retained strong (0.2-0.4) or even except exceptional (>0.5) OBs and DBs (FIG. 2b). It is unknown whether these cells represent a small age-resistant subsample, or if, instead, they would have exhibited even greater selectivities if studied before aging. The results for drifting bar and drifting grating stimuli were analyzed separately. Regardless of the type of stimulus used to study orientation and direction biases, old animals showed significantly decreased selectivity compared with young animals (p<0.001).

### EXAIPLE 6

### Selectivity Lost Due to Increased Response to Previously Non-Optimal Stimuli

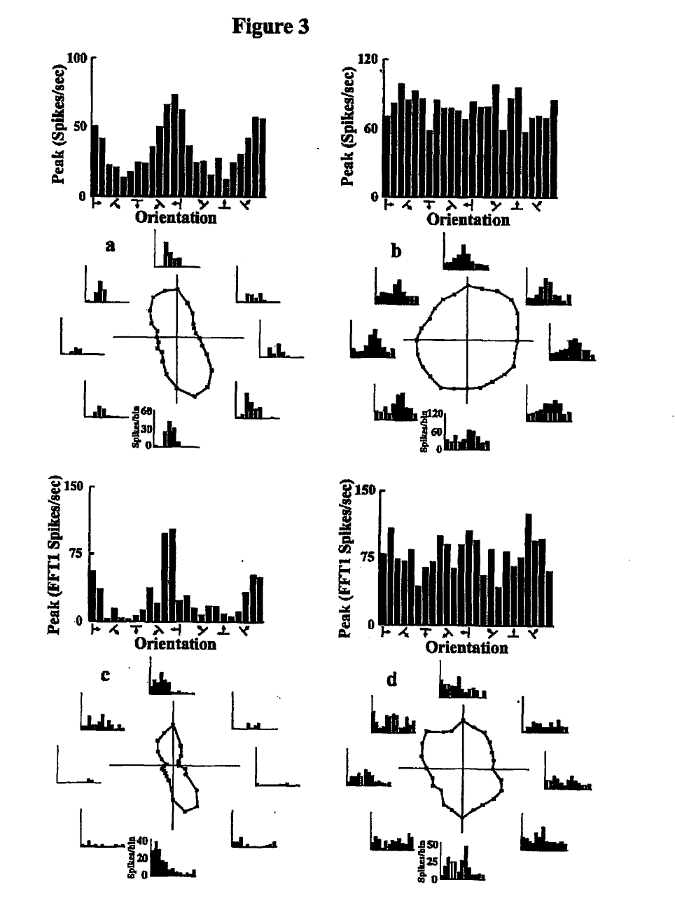

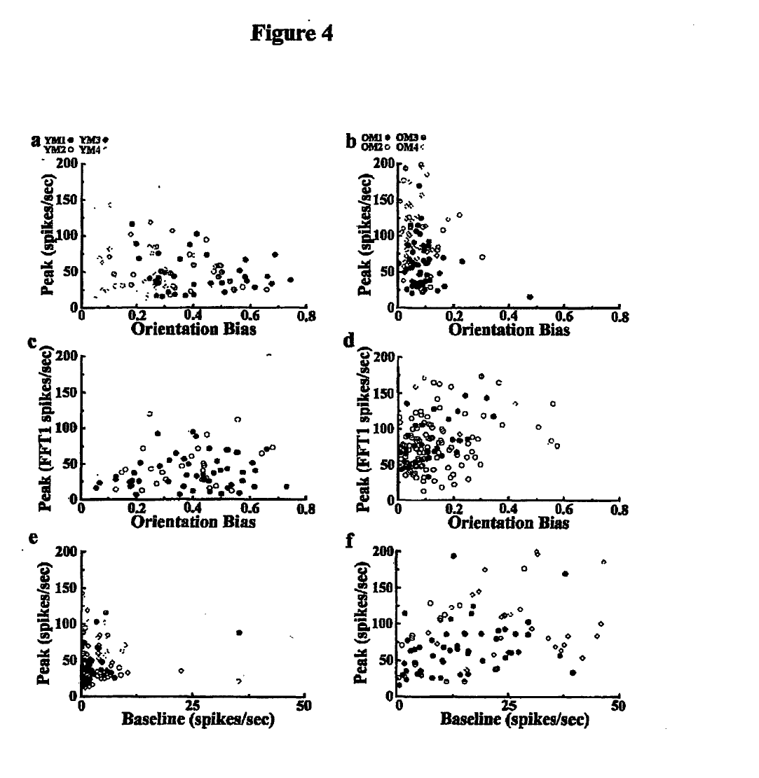

A reduction in orientation and direction biases could result from either an increased responsiveness to previously non-optimal orientations and directions, or from a reduced responsiveness to the previously optimal orientations and directions, or both. To assess these possibilities, the peak responses of young and old monkey cells to the drifting stimuli were used to compile tuning curves. If a substantial number of old monkey cells lost selectivity via reduced responses to the optimal stimulus alone, then the average peak response would be reduced in old compared with young monkeys. If old monkey cells instead lost selectivity via increased response to previously non-optimal stimuli, then the average peak response would be retained or increased in old monkeys. The latter result was obtained (FIGS. 3 and 4). Old monkey cells demonstrated increased peak responses to drifting luminance bars (FIG. 4b) and sinusoidal gratings (FIG. 4d) compared to young monkey cells (FIGS. 4a and c; p<0.05 in each case). Separate analyses compared the average peak responses for young versus old monkeys, and also showed a significant aging effect (luminance bar condition, Mann-Whitney test, p<0.05; sinusoidal grating condition, t-test, p=0.057). The increased amplitudes and decreased biases observed indicated that most cells in old animals responded strongly and reliably to all orientations and directions (FIG. 3). The peak amplitudes of the most selective old monkey cells (OB≧0.2) were also increased relative to young monkey cells (p<0.001). Therefore increased age leads to increased responsiveness to optimal and non-optimal stimuli alike.

The baseline response levels of neurons in old and young animals was also examined. V1 cells in old monkeys had a significant increase in spontaneous activity when compared with young animals (FIGS. 4e and f; p<0.001). A separate analysis compared the average baseline responses for young versus old monkeys and also showed a significant aging effect (Mann-Whitney test, p<0.05). Taken together, the increases in peak and baseline activity in old compared with young animals resulted in decreased peak-to-baseline (signal to noise) ratios in old animals (4.63; 7.8±9.5; median; mean±s.d.) compared with young (17.6; 27.2±27.2; p<0.001). A separate analysis compared the average peak-to-baseline ratios for young versus old monkeys and also showed a significant aging effect (Mann-Whitney test, p<0.05).

This data shows that the stimulus selectivity of single visual cortical neurons in the primates degrades with age, and that measurements of the stimulus selectivity can be used to assay for the effect of drugs that reverse the age related changes. V1 cells in the aged macaque showed significantly reduced orientation and orientation biases, accompanied by increased spontaneous and visually evoked activities. The general signaling capacity of cells in the old animals, judged by the signal-to-noise ratio, was reduced. The peak evoked-response data indicates that the reduced stimulus selectivity in old animals was accompanied by an increased responsiveness to optimal as well as non-optimal stimuli.

These findings explain, for the first time, why aged humans perform poorly at tasks requiring orientation discrimination and shape discriminations^{1,6-9}, which are believed to rely on the competence of orientation selective cells⁸⁻⁹ The ability to detect objects in motion is adversely affected by age^{1,2}. Performance of smooth pursuit eye-movement tasks that rely in part on motion detection also suffers with age¹⁷. Aged humans also demonstrate slowed reaction times to the onset of motion, and 20-40 ms of this delay is thought to be due to sensory degeneration. Cells in V1 are the first to show strong direction selectivity in macaque¹³ and approximately 25-35% of V1 cells are strongly direction selective¹⁵⁻¹⁶ (FIG. 2). The present results showed that the number of such cells is reduced in the aged macaque (FIG. 2, Table 1). Because V1 is the first site where strong orientation selectivity is observed in the macaques, losses at this site and/or extrastriate cortex are believed to mediate these perceptual declines.

### EXAMPLE 7

### Cortical and Subcortical Contributions to Aging Effects

Ophthalmological exams in the present study revealed no aberrations in retinal morphology or vasculature. In addition, neither examination of whole-mounted retina (FIGS. 1a-c) nor intracellular injection labeling of A (FIGS. 1d and e) and B ganglion cell revealed any substantial differences between old and young macaque retinal morphology. Since optical and retinal degeneration were minimal, then the physiological responses of retinal cells, LGNd cells and possibly even the geniculorecipient cell of V1 (found predominantly in layer 4) are relatively unaffected. Although anesthesia could have selective effects on aged subjects, in that case decreased response amplitudes to visual stimuli would have been observed in old animals rather than the increased amplitudes observed (FIG. 4).

It is believed that the decreased selectivity is predominantly due to changes in intracortical circuitry, and especially age-related loss of inhibitory function. The finding that cells in old monkeys showed increased spontaneous and visually driven activity, and were nonselective, particularly indicates a general degradation of inhibitory intracortical connections. This general degradation of inhibition has now been demonstrated to be due to an age-related effect on GABA-ergic connections, which can be reversed by the administration of a GABA-ergic agonist, as shown in the following Example.

### EXAMPLE 8

### Reversal of Age-Related Change with GABA-ergic Agents

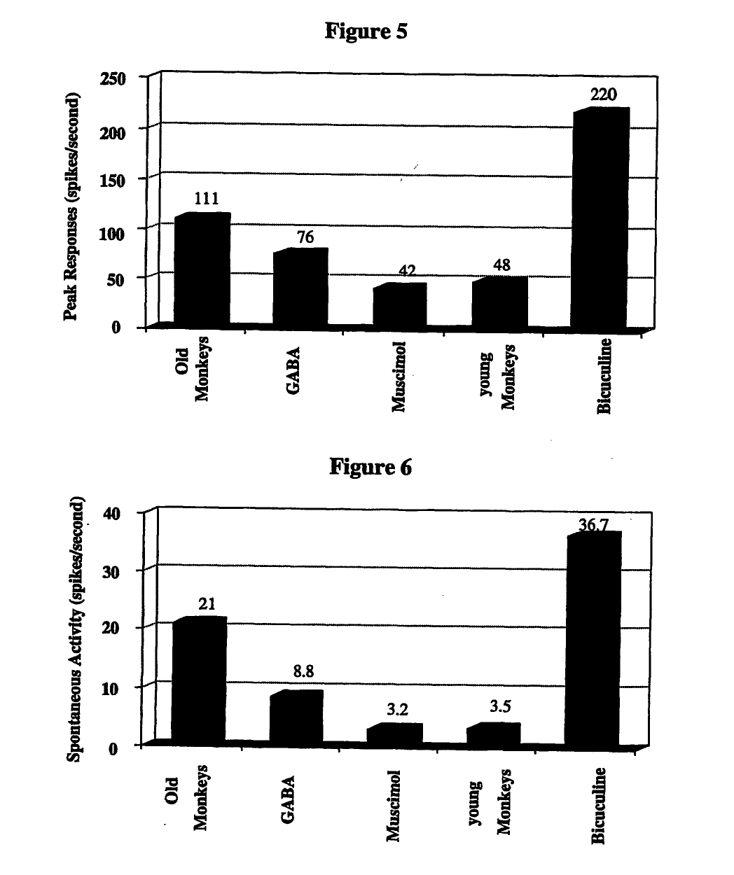

The maximum visually evoked responses of the V1 cortical cells were measured in untreated old monkeys, untreated young monkeys, and old monkeys treated with GABA, the GABA agonist muscimol, and the GABA antagonist bicuculline. The results, which are shown in FIGS. 5-9, show that cortical cells in old monkeys exhibit abnormally high peak responses (FIG. 5) and spontaneous activity (FIG. 6) compared to young monkeys. Bicuculline reduces GABA mediated inhibition, and further increases peak response (FIG. 5) and spontaneous activity (FIG. 6). GABA and the powerful GABA agonist muscimol increase GABA mediated inhibition and reduce the peak responses and spontaneous activity of cortical cells to the normal levels seen in young monkeys.

Extracellular action potentials of isolated cortical cells, LGNd cells and optic tract fibers were recorded with 3-5 MQ tungsten microelectrodes or microcapillary glass electrodes containing 4M NaCI. The electrode was advanced using a hydraulic microdrive (Kopf) or a piezoelectric microdrive (Burleigh Instruments) and was moved 50 to 75˜m between units to reduce sampling bias. Visual stimuli were generated on a Tektronix 608 display driven by a Picasso image synthesizer and specially designed texture/motion generator (Innisfree). The Picasso and texture/motion generator are controlled by computer (software package developed by Cambridge Electronics Design, LTD.). The system is able to randomly generate a broad spectrum of visual stimuli under computer control, collect the data, and perform on-line statistical analyses. In addition, the oscilloscope display can be moved to any point in the animal's visual field while at the same time maintaining a fixed distance between the display and the animal's retina. Thus, cells subserving all eccentricities (distance from fovea) can be observed without distortion.

The responses of the cells to the visual stimuli presented are stored in the computer for later analysis. The responses to the sinusoidal gratings are defined as the amplitude of the fundamental Fourier component of the post stimulus time histogram. For stimuli other than gratings the responses are defined as the peak response of the post stimulus time histogram with the total analysis time of 150-300 m/sec depending on the velocity of the drifting stimulus. Orientation and direction preferences and sensitivities are calculated for each cell using the statistical methods described elsewhere in detail (Batschelet, 1981; Leventhal et al., 1995; Thompson et al., 1989, 1994a, b; Wdrgotter et al., 1990; Zar, 1974).

Response strength, response variability, and times of neuronal modulation are determined for each spike train using an adaptation of the Poisson spike train analysis originally described by Legendy and Salcman (1985) and modified by Hanes et al. (1995) and Schmolesky et al. (1998). Since a distribution of interspike intervals (ISIs) approximates a Poisson distribution (Rodieck et al. 1962; Smith & Smith, 1965) this method provides a good null hypothesis to detect changes in neuronal modulation (Legendy and Salcman, 1985). The Poisson spike train analysis determines how improbable it is that the number of action potentials within a specific time interval is a chance occurrence.

A variety of visual stimuli can be generated by programming the graphics card (Stealth64 2001, Diamond Corp., New York). Visual patterns are displayed on a 5″ VGA monitor (Kristal Corp., St. Charles, EL) and imaged with a first-surface mirror (Edmund Scientific. Barrington, N.J.) and lens on the film plane of the microscope's camera port. This ensures that when the electrode tip is in focus in the eyepieces the visual stimulus is also focused on the retina.

The sensitivity of cells to spatial frequency is determined by testing the responses of cells to high contrast sinusoidal gratings of various spatial frequencies. The sensitivity of cells to stimulus contrast is determined by systematically testing the cells' responses to sinusoidal gratings of optimal spatial and temporal frequency. High contrast sinusoidal gratings of different temporal frequencies and optimal spatial frequencies are employed to determine temporal sensitivity.

In these experiments drugs were delivered through multibarreled micro-electrodes which had been positioned in the cortex as described in Example 1. The multibarrel electrode had an impedance of 5 M Ohm, and contained 0.1-0.5 M solutions of drug, which were administered by passing a current of 15-50 n amp for 1-3 minutes. Three barrels of the microelectrode held the drugs to be administered, and one barrel was filled with 4M NaCl in order to record the action potentials of the cells. Administration of one drug at a time was accomplished by passing current through the appropriate barrel. Holding current of 10 n amp is applied through the other barrels simultaneously in order to prevent leakage of the other drugs. The observed effects were seen three to five minutes after drug administration. The drug effects wear off five to ten minutes after drug administration ceases, and the cells in old animals revert back to their abnormal condition after drug administration ceases and GABA inhibition decreases. Intravenous drug application also improves cortical function in a similar way.

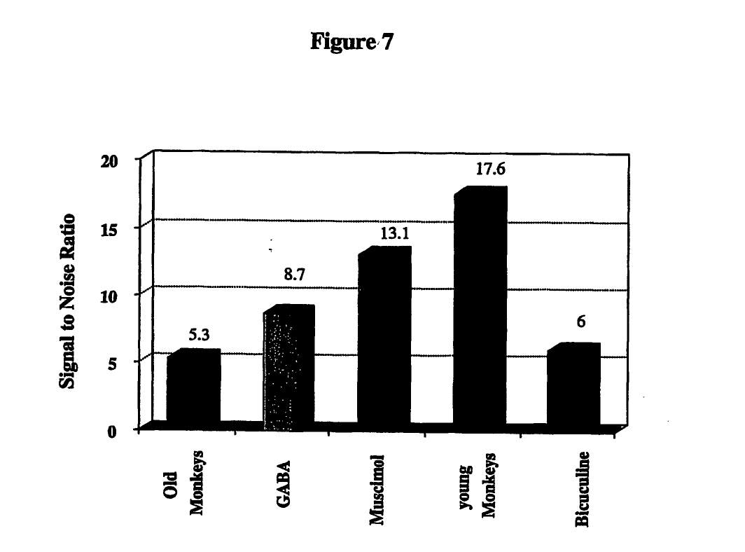

FIG. 7 shows the signal-to-noise ratios of cortical cells in old monkeys, young monkeys, and old monkeys treated with GABA and GABA agonists and antagonists. The signal-to-noise ratio of the cortical cells is the ratio of the response of the cell to appropriate stimuli, divided by the cell's spontaneous discharge rate. High signal-to-noise ratios allow cortical cells to function accurately and reliably. Low signal-to-noise ratios result in diminished cortical function throughout cerebral cortex. FIG. 7 shows that signal-to-noise ratios are abnormally low in old monkeys compared to young monkeys. The GABA antagonist bicuculline does not improve signal-to-noise ratios in old animals. In contrast, GABA, and especially the powerful GABA agonist muscimol, increased signal-to-noise ratios dramatically. This increase in signal-to-noise ratios will improve cortical function throughout cerebral cortex in old subjects.

Results similar to those presented here for the peak responses, spontaneous activities, and signal-to-noise ratios of cells in cortical area V1 can be generalized to other cortical areas such as V2, V3, V4, the medial temporal area, the medial superior temporal area (temporal lobe), frontal eye fields (frontal lobe), inferior temporal cortex (cognitive area) and others. In fact, V1 in man and old world monkeys sends inputs to over 30 separate areas of cerebral cortex in all lobes. Thus, changes observed in V1 in response to GABA-ergic agonist drugs will be reflected in the properties of cells in these areas. However, it is also possible to place the microelectrode of this Example in any of these brain areas, and confirm the effect of the drug by direct administration into that area of the brain. Alternatively, the drug can be given orally or by intravenous administration, and the effect recorded in the precisely positioned electrode.

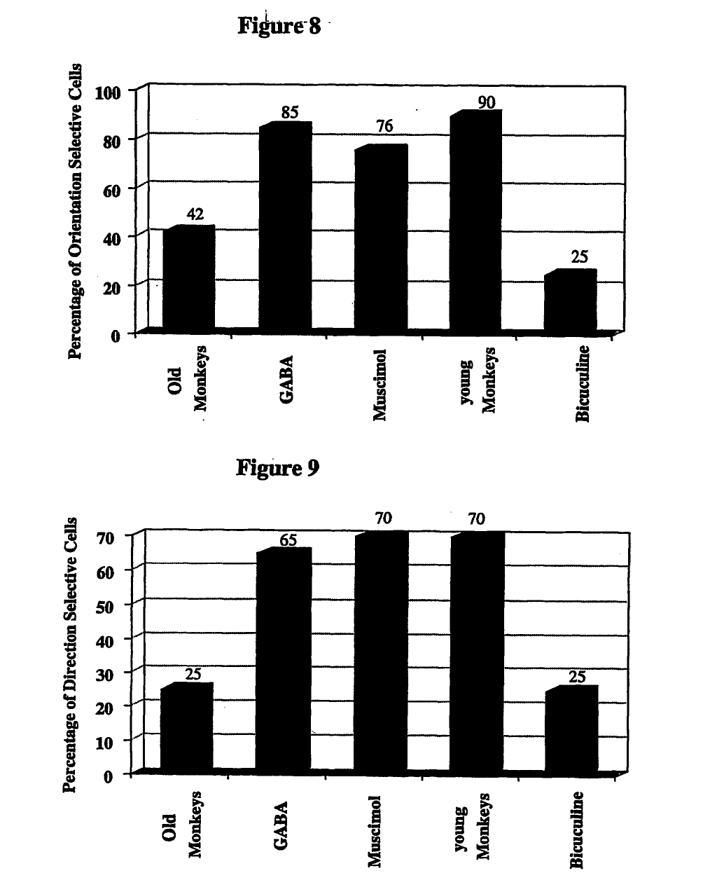

FIGS. 8 and 9 show the orientation (FIG. 8) and direction (FIG. 9) selectivity of cells in area V1 of old-world monkeys. Orientation and direction selective cells mediate the ability to perceive the shapes and directions of motion of objects. A reduction in the number of selective cells adversely affects visual perception. The ability to perform tasks such as driving a car are also be affected, because shape and direction discrimination are crucial in order to navigate through traffic. As illustrated in FIGS. 8 and 9, old monkeys exhibit a reduction in orientation and direction selective cells compared to young monkeys. The GABA antagonist bicuculline results in a further decrease in the number of selective cells.

In contrast, GABA and GABA agonists are capable of increasing the orientation and direction selective responses of cortical cells. Three to five minutes after intracortical delivery of GABA and GABA agonists, many cells that are unselective in old monkeys begin to exhibit clear orientation and direction selective responses. The result is that the proportion of selective cells in old monkeys treated with GABA and GABA agonists are very close to what is found in normal young monkeys.

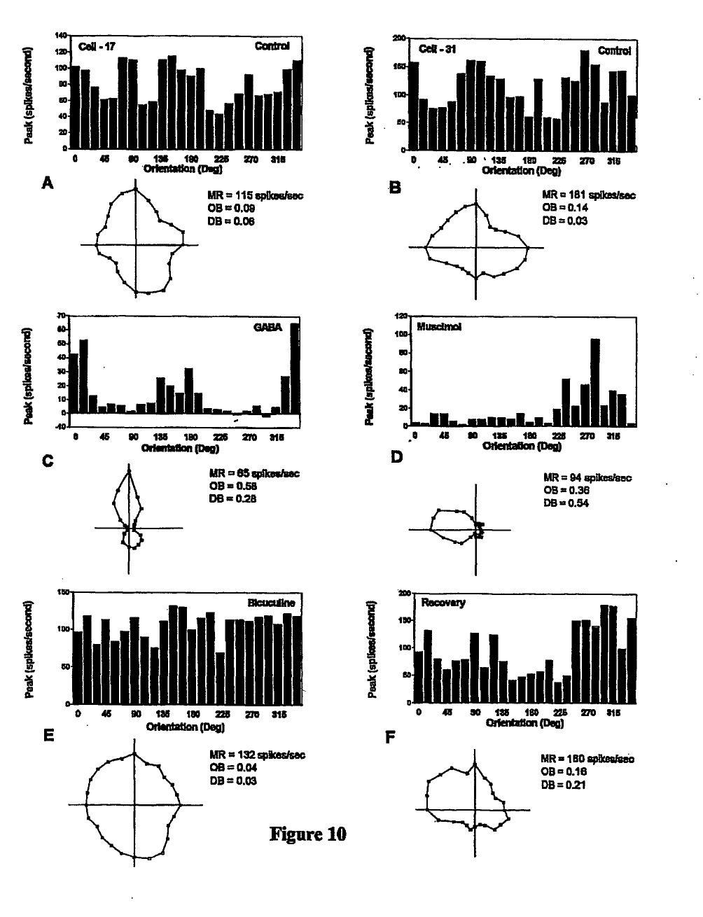

FIG. 10 shows the tuning curves and corresponding polar plots obtained for two representative cells in old world monkeys that received treatment with GABA, a GABA agonist (muscimol) and a GABA antagonists (bicuculline). Conventions are the same as in FIG. 3, in which the peak responses [MR], orientation biases [OB], and direction biases [DB] are shown for each condition. A typical cortical cell showing a lack of orientation and direction sensitivity is shown in (A). Three minutes following GABA application (C) this cell exhibited strong orientation and moderate direction selectivity. The cell's peak response decreased as did its spontaneous activity. GABA application was then discontinued and bicuculline application was begun (E). Within five minutes the cell lost its orientation and direction sensitivity and its peak response and spontaneous activity increased dramatically.

The responses of a second cell showing a degradation of orientation and direction selectivity in visual cortex of an old monkey is shown in (B). Three minutes following muscimol administration (D) this cell exhibited moderate orientation selectivity, very strong direction selectivity, a decreased peak response and decreased spontaneous activity. Five minutes after the discontinuation of muscimol administration the drug-induced improvement disappeared and the cells responses returned to the pre-drug condition (F).

### EXAMPLE 9

### Assays for Selecting Drugs to Treat Age-Related Cortical Dysfunction

The procedures described in Example 8 for studying cortical cells in old animals before, during, and after the administration of various drugs also provides new assays for finding agents that improve cortical function in the elderly. This testing can be used in all regions of cerebral cortex and will allow screening for drugs that will improve function of cortical areas involved in visual, auditory, somatosensory, motor, memory, language, analytical thought, language, and cognition.

In different areas of the brain, a battery of different tests can be applied to assess function in old animals and animals in which the various drugs and doses of drugs thought to affect GABA mediated inhibition are delivered. The electrodes can be placed in the specified locations of the cortex using the procedures described in Examples 1 and 2 for single neuron recording. Drugs being screened can be administered through the measuring microelectrodes as described in Example 8. Some specific examples of the projections to be tested are given below:

In visual cortex, function is assessed by testing for one or more of peak response, spontaneous activity, orientation selectivity, direction selectivity, signal-to-noise ratio, contrast sensitivity, and spatial frequency sensitivity. If testing spatial sensitivity, for example, a frequency of 40 cycles per second at arms length distance would be considered normal, while a frequency of 15 cycles per second would be considered low (and is a frequency that can be seen in older animals). The effect of administering GABA-ergic agents can be measured by determining whether the frequency after administration of the agent increases toward the “normal” value (such as 40 cycles per second).

In auditory cortex, function is assessed by testing the cell's frequency sensitivity, which screens for the ability to acquire sound.

In somatosensory cortex, function is assessed by the ability of cells to signal qualitatively different stimuli (temperature, pain, vibration, pressure) that are presented to the test subject. The ability of cells to signal the intensity of different stimuli (i.e. how hot, how hard, how fast the vibration) can also be determined.

In all areas the signal-to-noise ratios can be studied. In all cerebral cortical areas an improved signal-to-noise ratio will translate into improved function. The tests outlined above can be done in anesthetized, paralyzed animals using the techniques described in Examples 1 and 2. The drugs outlined in EXAMPLE 11 as well as other compounds that prove to have similar effects can be delivered through multibarreled microelectrodes while simultaneously recording the responses of the cells to various visual, auditory, and somatosensory stimuli. The improvements in the cell's function will be assessed. In general, if even one-fourth of the age-related decline in a property can be reversed, then clinical improvement would be expected, and the agent can be selected for further testing. Such further testing could involve, for example, administering the agent to an animal or human, followed by testing to assess one or more cortical functions, as described in greater detail in Example 10.

Multiunit recording techniques and/or cortical evoked potentials will also be useful in assessing drug effects. For example, microelectrodes can be positioned in multiple cortical areas, such as visual cortex, somatosensory cortex, and or auditory cortex, and responses simultaneously measured from each of these areas. Single or multiple recordings can be performed while administering one or more test agents through the microelectrodes, or while systemically administering the one or more test agents.

### EXAMPLE 10

### Subsequent Testing of Compounds

Drugs that result in significant improvement at the single cell level are selected for further testing. An example of such further testing is to administer them orally or by injection to old and young awake, behaving monkeys that are trained to perform a variety of different sensory and motor tasks. These tasks can include visual discrimination (for example discriminating lines of different orientation and direction), auditory discrimination (for example discriminating different frequencies), somatosensory discrimination (for example trained to respond to differences in pressure, temperature, vibration), motor tasks (such as rapidly assembling blocks), cognitive discrimination (such as choosing a unique shape in a complicated background, for example “find Waldo”). The behavioral training techniques to carry out the studies exist and are described below.

Visual tasks have been used as an example of one technique for selecting agents that improve cortical function, but virtually all aspects of higher cortical function can be investigated in monkeys (or humans) in this way. The usefulness of this approach in test animals is that it allows one to determine the functional improvement that results from the agents selected for further investigation.

When testing the ability of old and young monkeys to discriminate orientation and direction, spatial frequency and contrast sensitivity may also be studied. Animals are tested before, during, and after the intravenous administration of the various types of drugs to be tested. Other modes of administration can of course be used, but intravenous adinistration is described in this example because of its more predictable bioavailability, and more rapid onset of action.

Monkeys are restrained in a primate chair, and their heads positioned so that they must look straight ahead. They view two Tektronix 608 oscilloscopes simultaneously. Stimuli are generated in the same fashion and at the same distance as during single unit recordings. Thus, behavioral results will be directly comparable to physiological ones. Monkeys are trained to touch a touch pad to signal a correct choice, and correct choices are rewarded by administering food.

Orientation discrimination is studied by first training the monkey to discriminate between a matched condition (two high contrast high spatial frequency gratings where both are horizontal), and a non-matched condition (two identical gratings where one grating is vertical and the other is horizontal). Gratings are flashed on for two seconds, and the animal has a total of three seconds from stimulus onset to respond. This timing is altered as needed to assure that old animals can easily complete the task. An equal number of matched and non-matched trials may be randomly interleaved. The animal is rewarded for responding to the non-matching condition, and for not responding to the matching condition. Once this task is learned (i.e. saturation of percentage correct decisions) the orientation of the vertical grating is changed and the difference between the two gratings in the non-matching condition is decreased in 5-degree increments with training before each increment. When the monkey fails to reach peak performance (as compared to the initial test phase) the increments are reduced to one degree and testing continues until threshold (the disappearance of the ability to distinguish between the two conditions) is determined.

Direction sensitivity is studied similarly with moving spots. Animals are first trained to discriminate between a matching condition (two high contrast, one degree spots moving horizontally in the same direction) and a non-matching condition (one spot moving vertically and the other moving horizontally). The direction difference is then decreased as described above, until the ability to discriminate between the directions is lost.

Spatial frequency sensitivity is studied as above with flashing gratings where the non-matched condition is one sinusoidal grating of one cycle/degree, and a blank screen of equal size and overall luminance and the matched condition is two blanks screens. After saturation the spatial frequency of the grating will be increased in one-cycle/degree increments until peak performance deteriorates. Then the increments will be decreased to 0.1 cycles/degree until threshold is reached. A value of 40 cycles per degree is usually normal, whereas 15 cycles per second is a lower spatial frequency sensitivity. Hence an increase of spatial frequency sensitivity (for example from 15 toward 45 cyles per degree) would be an indication that an agent has improved sensitivity.

Contrast sensitivity is studied as above, where the non-matching condition consists of one high contrast sinusoidal grating of one cycle/degree, and a blank field of equal size and luminance, and the matched condition is two blank screens. After learning has saturated, the contrast is then decreased (for example in increments of 0.1) until peak performance deteriorates. Increments are then decreased (for example in 0.01 increments) until threshold is reached. The foregoing tasks were designed to be as stress free as possible to assure that old animals will have no problem learning them. in all cases the monkeys simply have to determine whether the two screens differ either in orientation, direction, contrast or spatial frequency. Thus, all tasks are of the simple go (hit the touch pad during non-matching condition) no-go (do not hit the pad during the matching condition) variety. This task does not require rigid head restraint, and the setup employed has been used even to study lesioned animals.

It is particularly useful if the monkeys used are prescreened to have normal optics and be in good health. These animals exhibit quite normal behavior. Both old and young animals can be tested successfully using this approach.

The apparatus is designed so that monkeys are trained by food reward to enter a primate chair, so that they push their head up and through an adjustable hole at the top of the compartment. Adjustable molded plastic baffles are attached at the sides and the back of the head to prevent large head movements. The entire compartment rests on a mobile trolley, which is placed in front of the two visual display units. When the sliding front of the compartment is removed the animals can reach out and touch the monitor and retrieve a food reward.

All animals are tested individually so that inter-animal variability can be assessed. In addition, several statistical techniques have been designed specifically to analyze distributions of angles (circular statistics), and are used to help interpret the data. A complete account is found in Batschelet (1981). Behavioral results are analyzed using appropriate statistics based upon signal detection theory (MacMillan and Creehman, 1991; Thompson et al., 1996).

Although this Example discussed testing agents for further study in primate models, it is also possible to test changes in cortical function in humans who have been administered the test agents. In recent years, a variety of tests have been developed to study the age-related decline in visual function that accompanies normal aging, Alzeimer's disease, and Parkinson's disease. For example, the Cambridge Neurophysical Test Automated Battery (CANTAB-13 tests and Neurotouch-16 tests) is used either for the initial diagnosis of an age-related decline in cortical function. There are CANTAB batteries applicable to virtually all aspects of neural deficits that accompany normal and pathological aging. These test batteries provide an excellent tool for selecting subjects who are in need of treatment with GABA-ergic treatments, or for functionally monitoring the effect of a GABA-ergic test agent upon neural function in the elderly.

The CANTAB battery and/or other tests of cognitive functioning can be applied to every subject in every step of cognitive decline. They can be applied to study the effects of various drugs in all stages of age-related decline.

The test and treatment methods described in these examples are useful for a variety of age-associated disorders of cortical (for example cortical) decline in the elderly. These “age-associated” disorders of cortical decline extend on a continuum from normal age-related senescence to severe dementias associated with Alzheimer's disease and Parkinson's disease in an aging population.

### EXAMPLE 11

### Examples of Compounds for Treatment and Screening

A variety of GABA-ergic agents (agents which increase GABA-mediated effects) can be used in the treatment of age-related disorders brought about by cortical decline. Many likely test agent candidates are also available. Examples of such agents include agents which inhibit GABA aminotransferase, such as vigabatrin; agents which inhibit GABA transferase and succinyl aldehyde, such as valproate, valproic acid, and divalproex (or their pharmaceutically acceptable salts); agents which facilitate GABA receptors, such as topiramate; agents which block GABA uptake into presynaptic neurons, thus permitting more GABA to be available for binding, such as tigabine and its pharmaceutically acceptable salts, such as tigabine hydrochlroide; agents which facilitate GABA-A mediated inhibition, such as benzodiazepines, which enhance GABA effects without directly activating GABA receptors, and/or which increase the frequency of chloride channel openings; agents which facilitate GABA-A mediated inhibition duration of GABA gated channel openings, such as barbiturates; GABA-A receptor binding agonists at the BZ1 (omega 1) receptor subtype, such as imidazopyradines; agents which facilitate GABA-B mediated inhibition, such as baclofen; and agents which facilitate GABA-C mediated inhibition, such as caca.

Examples of GABA agonists and GABA facilitators that are useful in the disclosed methods are shown in Table 2 (GABA Drugs). Examples of GABA antagonists (that can be used to offset undesired effects of the GABA agonists) include the drugs shown in Table 3 (GABA Antagonists). The class of benzodiazepine drugs is discussed in Principles of Pharmacology (Munson ed.), Chapman & Hall, 1995 in chapter 14 (and particularly at pages 246-247)(chapter 14 is incorporated by reference). Examples of some benzodiazepines are also shown in Table 4 (List of 33 Benzodiazepines).

The GABA-ergic drugs (which mediate GABA effects, and include GABA agonists) can be used in combination with a variety of other drugs. For example, the GABA-ergic drugs can be used in combination with GABA antagnoists, which are useful in reducing any side effects of treatment with GABA agonists such as a benzodiazepines. Alternatively, the GABA-ergic drugs can be used in combination with already available cognition enhancing drugs, such as Cognex (tacrine hydrochloride). In other embodiments, two or more of the GABA-ergic drugs can be used in combination, for example drugs which mediate GABA-ergic activity by different mechanisms (for example one agent that facilitates GABA receptors and another agent that inhibits GABA-aminotransferase).

Any of the GABA-ergic agents that are found to enhance cortical function can be provided in a unit dosage form, for example in combination with a pharmaceutically suitable carrier.

A number of other substances can be used, including excitatory agents, which affect GABA levels in the brain. Such agents can work either directly or indirectly, and include glutamate, AMPA (2-amino-3-hydroxy-5-methyl-4-isoxazole propionic acid), kainate, endogenous zinc, and others. Binding sites for these substances are found throughout visual cortex. Non-NMDA AMPA receptor antagonists such as CNQX (6-cyano-7-nitroqunoxaline-2,3-dione), DNQX and NBQX (see Pharmacol. Biochem. Behav. 51:153-158, 1995) are potential agents that can be tested in accordance with the techniques disclosed in this specification, and/or used to treat the age-related loss of GABA-ergic pathways. AMPA receptor antagonists that are candidate agents include (S)-5-fluorowillardine; 1-(quinoxalin-6-ylcarbonyl)piperidine (CX-516); (S)-2,3-dihydro-[3,4]cyclopentano-1,2,4-benzothiadiazine-1,1-dioxide. Other candidate agents include those that block excitatory responses at the AMPA receptor, for example agents such as phenobarbital; and topiramate.

Agents described in this example are suitable for screening in the present method, and at least some of them would be useful in the treatment of sensory, motor, and cognitive declines that accompany old age. They and their analogs can be screened for such uses with the techniques described in this specification.

### EXAMPLE 12

### Pharmaceutical Compositions

The invention also contemplates various pharmaceutical and laboratory compositions that improve cortical function. When the agent is to be used as a pharmaceutical, the agent is placed in a form suitable for therapeutic administration. The agent may, for example, be included in a pharmaceutically acceptable carrier such as excipients and additives or auxiliaries, and administered to a subject. Frequently used carriers or auxiliaries include magnesium carbonate, titanium dioxide, lactose, mannitol and other sugars, talc, milk protein, gelatin, starch, vitamins, cellulose and its derivatives, animal and vegetable oils, polyethylene glycols and solvents, such as sterile water, alcohols, glycerol and polyhydric alcohols. Intravenous vehicles include fluid and nutrient replenishers. Preservatives include antimicrobial, anti-oxidants, chelating agents and inert gases. Other pharmaceutically acceptable carriers include aqueous solutions, nontoxic excipients, including salts, preservatives, buffers and the like, as described, for instance, in Remington's Pharmaceutical Sciences, 15th ed., Easton: Mack Publishing Co., 1405-1412, 1461-1487, 1975, and The National Formulary XIV., 14th ed., Washington: American Pharmaceutical Association, 1975). The pH and exact concentration of the various components of the pharmaceutical composition are adjusted according to routine skills in the art. See Goodman and Gilman The Pharmacological Basis for Therapeutics, 7th ed.

The methods disclosed herein involve administering to a subject a therapeutically effective dose of a pharmaceutical composition containing the compounds of the present invention and a pharmaceutically acceptable carrier. The administration of the pharmaceutical composition of the present invention may be accomplished by any means known to the skilled artisan (for example, intravenous, subcutaneous, intra-peritoneal, topical, intra-nasal, or oral administration).

The pharmaceutical compositions are preferably prepared and administered in dose units. Solid dose units are tablets, capsules and suppositories. For treatment of a patient, depending on activity of the compound, manner of administration, nature and severity of the disorder, age and body weight of the patient, different daily doses are necessary. Under certain circumstances, however, higher or lower daily doses may be appropriate. The administration of the daily dose can be carried out both by single administration in the form of an individual dose unit, or in several smaller dose units, and also by multiple administration of subdivided doses at specific intervals.

Initial dosage ranges can be selected to achieve an inhibitory concentration in target tissues that is similar to in vitro inhibitory tissue concentrations. The dosage is ideally not so large as to cause adverse side effects, such as unwanted cross-reactions, anaphylactic reactions, and the like. Generally, the dosage will vary with the age, condition, sex, and extent of the disease in the patient and can be determined by one skilled in the art. The dosage can be adjusted for each individual in the event of any contraindications and can be readily ascertained without resort to undue experimentation. In any event, the effectiveness of treatment can be determined by monitoring the subject's status on a neurocognitive test, such as the CANTAB battery. However, any neurological function test can be used to assess cortical function, including repeating lists of items, reporting biographical information (such as one's own telephone number), or responses to questions about current events (such as the name of the President of the United States).

The pharmaceutical compositions according to the invention are generally administered intravenously, orally or parenterally, or as implants. Suitable solid or liquid pharmaceutical preparation forms are, for example, granules, powders, tablets, coated tablets, (micro)capsules, suppositories, syrups, emulsions, suspensions, creams, aerosols, drops or injectable solution in ampule form and also preparations with protracted release of active compounds, in whose preparation excipients and additives and/or auxiliaries such as disintegrants, binders, coating agents, swelling agents, lubricants, flavorings, sweeteners or solubilizers are customarily used as described above. The pharmaceutical compositions are suitable for use in a variety of drug delivery systems. For a brief review of present methods for drug delivery, see Langer, Science, 249:1527-1533, 1990, which is incorporated herein by reference. The pharmaceutical compositions may be administered locally or systemically.

The pharmaceutical compositions of the invention include chemical compounds, peptides, and peptidomimetics. When co-administered in combination with one or more other drugs useful in the treatment of cortical decline, the compounds may be administered by either concurrent or sequential administration of the active agents.

In view of the many possible embodiments to which the principles of the invention may be applied, it should be recognized that the illustrated embodiments are only particular examples of the invention and should not be taken as a limitation on the scope of the invention. We therefore claim as our invention all that comes within the scope and spirit of the following claims.

### REFERENCES

The following references are incorporated by reference as fully as if they appeared herein. Also incorporated by reference is Schmolesky et al., Degradation of stimulus selectivity of visual cortical cells in senescent rhesus monkeys, Nat Neurosci. 2000 Apr;3(4):384-90.

1. Kline, D. W. & Schieber, F. in Handbook of the Psychdology of Aging (eds. Birrcn, J. E. & Schaie, K. W.) 296-331 (Van Nostrand, New York, 1985).

2. Kline D. Light. aging and visual performance. Vision Vis. Dys. 16, 150-161 (1991).

3. Tran, D. B., Silverman, S. E., Zimmerman, K. & Feldon S. E., Age-related deterioration of motion perception and detection. Gracfc's arch. Clin. Exp. Ophthalmol. 236, 269-273 (1998).

4. Porciatti V., Fiorcentini A., Morrane, M. C., & Burr, D. C. The effects of aging on reaction times to motion onset. Vis. Res. 39, 2157-2164 (1999).

5. Kline D. W., Schieber. F., Abusamra, L. C. & Coyne, A. C. Age and the visual channels: Contrast sensitivity and response speed. J. Gerontol. 38, 211-216 (1983).

6. Weston, H. C. The effect of age and illumination upon visual performance with close sights. Br.J. Ophthalmol. 32, 645-653 (1948).

7. Ordy J. M. & Brizzee K. R in Sensory Systems and Communication in the Elderly (eds. Ordy J. M. & Brizzee, K. R.) 13-50 (Raven, N.Y., 1979).

8. Owsley, C., Sekuler, R. & Boldt, C. Aging and low-contrast vision: face perception. Invest. Ophthalmol. Vis. Sci. 21, 362-359 (1981).

9. Owsley, C. & Sloane., M. E Contrast sensitivity, acuity, and the perception of ‘realworld’ targets. Br. J. Ophthalmol. 71, 791-796 (1987).

10. Spear, P. D. Neural basis of visual deficits during aging. Vis. Res. 33, 2589-2609 (1993).

11. Ahmad, A. & Spdar, P. D. Effects of aging on the size, density, and number of rhesus monkey lateral geniculate neurons. J. Comp. Neurol. 334, 631-643 (1993).

12. Spear, P. D., Moore, R. J., Kim, C. B. Y., Xue, J. & Tumoss, N. Effects of aging on the primate visual system: Spatial and temporal processing by lateral geniculate neurons in young adult and old rhesus monkeys. J. Neurophysiol. 72, 402-420 (1994).

13. Hubel, D. H. & Wiesel T. N. Receptive fields and functioned architecture of monkey striate cortex. J. Physiol. (Lond) 195, 215-243 (1968).

14. Orban, G. A. in The Neural Basis of Visual Function (ed. Leventhal, A.G.) 173-217 (CRC Boca Raton, Fla., 1991).

15. Schiller, P. H., Finlay, B. H & Volman, S. E. Quantitative studies of single-cell properties in monkey striate cortex. I. Spatiotemporal organization of receptive fields. J. Neurophysiol. 39, 1288-1319 (1976).

16. DeValois, R. L., Yund, E. W. & Hepler, N. The orientation and direction selectivity of cells in the macaque visual cortex. Vis. Res. 22, 531-544 (1982).

17. Ross, R. G. et al. The effects of age on smooth pursuit tracking task in adults with schizophrenia and normal subjects. Biol. Psychiatry 46,384-391 (1999).

18. Porciatti, V., Burr D. C., Morrone, C. & Fiorentini, A. The effects of aging on the pattern electroretinogram and visual evoked potential in humans. Vis. Res. 32, 1199-1209 (1992).

19. Tomoda, H., Celesia G. C., Brigell, M. G. & Toleikis, S. The effects of age on steady-state pattern electroretinograms and visual evoked potentials. Doc. Ophthalmal. 77, 201-211 (1991).

20. Trick, G. L., Trick, L. R. & Haywood, K M. Altered pattern evoked retinal and cortical potentials associated with human senescence. Curr. Eye Res. 5, 717-724 (1986).

21. Leventhal, A. G., Rodieck, R. W. & Dreher, B. Retinal ganglion cell classes in the Old World monkey: Morphology and central projections. Science 213, 1139-1142 (1981).

22. Leventhal, A. G., Ault, S. J. & Vitek, D. L. The nasotemporal division in primate retina: the neural bases of macular sparing and splitting. Science 240, 66-67 (1988).

23. Leventhal, A. G., Thompson K. G. & Liu, D. Retinal ganglion cells within the foveola of new world (Saimiri sciureus) and old world (Macaca fascicularis) monkeys. J. Comp. Neurol. 338, 242-254 (1993).

24. Perry, V. H. & Covey, A. The morphologic correlates of X- and Y-like retinal ganglion cells in the retina of monkeys. Exp. Brain Res. 43, 226-228 (1981).

25. Watanabe, M. & Rodieck, R. W. Parasol and midget ganglion cells of the primate retina. J. Comp. Neurol. 289, 434-454 (1989).

26. Sato, H. Katsuyama, N. Tamura, H., Hata, Y. & Tsumoto, T., Mechanisms underlying orientation selectivity of neurons in the primary visual cortex of the macaque. J. Physiol. (Lond.) 494, 757-771 (1996).

27. Eysel, U. T., Shevelev, I. A., Lazareva, N. A., Sharaev, G. A. Orientation tuning and receptive field structure in cat striate neurons during local blockade of intracortical inhibition. Neuroscience. 84, 25-36 (1998).

28. Sillito, A. M. Inhibitory processes underlying the directional specificity of simple, complex, and hypercomplex cells in the cat's striate cortex. J. Physiol. (Lond.) 271, 699-720 (1977).

29. Sillito, A. M. Inhibitory mechanisms influencing complex cell orientation selectivity and their modification at high resting discharge levels. J. Physiol. (Lond.) 289, 33-53 (1979).

30. Volgushev, M., Pei, X., Vidyasagar, T. R. & Creutzfeldt, O. D. Excitation and inhibition in orientation selectivity of cat visual annex neurons revealed by whole-cell recordings in vivo. Vis. Neurosci. 10, 1151-1155 (1993).

31. Crook, J. M., Kisvarday, Z. F. & Eysel, U. T. Evidence for a contribution of lateral inhibition to orientation tuning and direction selectivity in cat visual cortex: reversible inactivation of functionally characterized sites combined with neuroanatomical tracing techniques. Eur. J. Neurosci. 10, 2056-2075 (1998).

32. McGeer, E. & McGeer, P. in Neurobiology of Aging (eds. Terry, R. D. & Gershon, S.) 389-403 (Raven, N.Y., 1976).

33. Dustman, R. E., Emmerson, R. Y. & Shearer, D. E. Life span changes in electrophysiological measures of inhibition. Brain Cogn. 30, 109-126 (1996).

34. Scheibel, M., Lindsay, R., Tomiyasu, U. & Scheibel, A. Progressive dendritic changes in aging human cortex Exp. Neurobiol. 47, 392-403 (1975).

35. Vincent, S. L., Peters, A. & Tigges, J. Effects of aging on the neurons within area 17 of rhesus monkey cerebral cortex. Anat. Rec. 223, 329-341(1989).

36. Samoraiski T. Central neurotransmitter substances and aging: A review. 1. Am. Gcr. Sac 25 337-348 (1977).

37. Peters A. losephson K. & Vincent S. L. Ethers of aging on the neurall cells and pericytes within area 17 of the rhesus monkey cerbral cortex Anat. Red 229 384-398 (1991).

38. Peters A. & Snhares' C. Aging and the Meynan calls in rhesus monkey primary visual annex. Anat. R″ 236 721-729 (1993).

39. Kim C. B. Y. Pier L P. & Spear R D. Effects of aging on numbers and sizes of neurons in histochemically defined subregions of monkey striate annex. Anat. Rec. 247 119-128 (1997).

40. Priers A. et al., A further evaluation of the effect of age on striate cortex of the rhesus monkey. Neurobiol. Aging 18 29-36

41. Peers A. Morrison I. H. Rosene D. L & Hyman B. T. Are neurons lost from the primate cerebral cortex during normal aging? Cereb. Cortex 8 295-300 (1998).

42. Fitzpatrick et al., Distribution of GABAergic neurons and axon terminals in the macaque striate annex. 1 Comp. Neurol 264, 7i91 (1987).

43. Leventhal A. G. & Hirsch H. V. B. Receptive-field properties of different classes of neurons in visual cortex of normal and dark-reared cats. 1 Neurophysiol. 43 1111-1132 (1980).

44. Sherman S. M. & Spear P. D. Organization of visual pathways in normal and visually deprived cats. Physiol Rev. 62 735˜855 (1982).

45. Tigges et al., Survival rate and life span of rhesus monkeys n the Yerkes Regional Primate Research Center. Amer. J. Primatol., 15, 263-273 (1988).

46. Walker E R Mammals of the World (Johns Hopkins Univ. Press Baltimore)

47. SchmolerJry M. T. et al., Signal ting across the macaque visual tandem. 1 Neurophysiol. 79 3272-3278 (1998).

48. Leventhal A. G. Thompson K G Liu. D. Zhou Y. & Ault S. 1. Concomitant sensitivity to orientation direction and color of cells in layers 2,3 and 4 of monkey striate cortex. J. Neurosci. 15, 1808-1818 (1995).

49. Pu et al., A method for fast and stable intracellular staining of retinal ganglion cells. J. Neurosci. Methods 41, 4549 (1992).

50. Pu M. Dendritic morphology of cat retinal ganglion cells projecting to suprachiamatic nucleus. J. Comp. Neurol. 414 267-274 (1999).

51. Eysel, U. T., Shevelev, I. A., Lazareva, N. A., Sharaev, G. A. Orientation tuning and receptive field structure in cat striate neurons during local blockade of intracortical inhibition. Neuroscience. 84, 25-36 (1998).

52. Sato, H., Katsuyama, N., Tamura, H., Hata, Y. & Tsumoto, T. Mechanisms underlying orientation selectivity of neurons in the primary visual cortex of the macaque. J. Physiol. (Lond.) 494, 757-771 (1996).

53. Sillito, A. M. GADA mediated inhibitory processes in the function of the geniculo-striate system. Prog. Brain Res., Mize, R. R., Marc, R. E., Sillito, A. M. (Eds.) 90, 349-384 (1992).

54. Sillito, A. M., Kemp, J. A. & Blakemore, C. The role of GABAergic inhibition in the cortical effects of monocular deprivation. Nature. 291, 318-320 (1981).

55. Sillito, A. M. The contribution of inhibitory mechanisms to the receptive field properties of neurons in the striate cortex of the cat. J. Physiol. 250, 305-320 (1975).

56. Sillito, A. M. The effectiveness of bicuculline as an antagonist of GABA and visually evoked inhibition in the cats striate cortex. J. Physiol. 250, 287-304 (1975).

TABLE 2
GABA DRUGS

```
  GABA (gamma-aminobutyric acid) is the most important inhibitory
neurotransmitter in the CNS. By gating negative chloride (Cl⁻) ions into
the interior of nerve cells, GABA inhibits the presynaptic release of
neurotransmitter due to a positive voltage polarization pulse. Such
inhibition is extremely common: GABA receptors can be found at 60-80%
of CNS neurons.
  Subtypes of GABA receptors can be activated by the mushroom toxin
muscimol (at the A subtype) as well as the antispasmodic amino acid
baclofen (B subtype). These drugs directly mimic the action of GABA at
the receptor.
  Allosteric facilitation of GABA receptors occurs at several distinct
sites: the compounds which bind there are used as sedatives and
anxiolytics. These compounds bend the receptor open to indirectly
facilitate GABA binding.
```

GABA agonists/facilitators

Muscimol
Progabide
Riluzole
Baclofen
Gabapentine (Neurontin)
Vigabatrin
Valproic acid (Depakote)
Tiagabine (Gabitril)
Lamotrigine (Lamictal)
Phenytoin (Dilantin)
Carbamazepine (Tegretol)
Topiramate (Topamax)

```
  Progabide is a pro-drug which decomposes to GABA in the CNS. It
crosses the blood-brain barrier, which GABA itself being a zwitterion
(doubly-ionized amino acid), does not. Vigabatrin (gamma-vinyl-GABA)
inhibits GABA-aminotransferase (GABA-T), the enzyme responsible for
degrading GABA in the synapse. It thus prolongs the sojourn of GABA
molecules and promotes binding in this way.
  Depakote (valproic acid) seems to act on nerve membranes to reduce
susceptibility to seizure. At high doses it acts like vigabatrin to inhibit
GABA-T. Gabapentine is another recently marketed antiepileptic
(Neurontin) that is also finding psychiatric application as a mood
stabilizer. The neurological rationale for this application is that panic
attacks (or mania in bipolar disorder) resemble epilepsy in that they are
characterized by a pre-panic “kindling” phenomenon, characterized by
repetitive neural firings, leading to a critical stage. Gabapentine may
encourage production of or discourage degradation of GABA Lamotrigino
probably works by reducing release of glutamate, an excitatory neuro-
transmitter usually governed by the inhibitory GABA.
  Novel GABA drugs represent one of the most active areas of psycho-
tropic research. Riluzole, for instance, is a GABA uptake inhibitor with
anticonvulsant and hypnotic properties; it also blocks sodium channels and
inhibits glutamate release.
```

TABLE 3
GABA ANTAGONISTS

Flumazenil
Bicuculline
Amiphenazole
Beta-CCB
Beta-CCE
Harmaline
Picrotoxinin
Picrotin
Tutin
Hyenanchin

```
  Flumazenil is a benzodiazepine which binds to the GABA
receptor at the benzodiazepine site without deforming it so as to enhance
GABA binding. It is thus a competitive antagonist to the benzodiazepine
sedatives. Bicuculline is a selective GABA-A antagonist directly at the
site where GABA binds.
  By contrast the beta-carbolines (CCE, CCB and CCM) are mild inverse
agonists, i.e. they not only bind to and block the benzodiaepine site on
the GABA receptor, but modify the receptor function to decrease GABA
activity. They also show strong though ephemeral MAO-inhibiting ability.
A structural extension of serotonin, chemical variants of the beta
carbolines (tetrahydro forms) have been detected in human urine and milk.
They occur more plentifully in various herbs, particularly passion flower,
yage, B. caapi, and other herbs. Harmala species are high in beta-
carbolines like harmaline.
  The picrotoxin group of toxins are naturally-occurring GABA antagonist
which can cause death due to convulsions. Tutin is present in some forms
of poison honey.
```

TABLE 4
Annex A - List of 33 benzodiazepines and 8 other substances
The substances currently listed in Schedule 4 Part II of the Misuse of
Drugs Regulations 1985 include 33 benzodiazepines and 8 other
substances. They are listed below:
33 benzodiazepines

| | |
|---|---|
| Alprazolam | Haloxazolam |
| Bromazepam | Ketazolam |
| Brotizolam | Loprazolam |
| Camazepam | Lorazepam |
| Chlordiazepoxide | Lormetazepam |
| Clobazam | Medazepam |
| Clonazepam | Midazolam |
| Clorazepic acid | Nimetazepam |
| Clotiazepam | Nitrazepam |
| Cloxazolam | Nordazepam |
| Delorazepam | Oxazepam |
| Diazepam | Oxazolam |
| Estazolam | Pinazepam |
| Ethyl loflazepate | Prazepam |
| Fludiazepam | Tetrazepam |
| Flurazepam | Triazolam |
| Halazepam | |

8 other substances

Aminorex
N-Ethylamphetamine
Fencamfamin
Fenproporex
Mefenorex
Mesocarb
Pemoline
Pyrovalerone

We claim:

1. A method of treating a subject having age-related decreases in cortical function, comprising administering to the subject a therapeutically effective amount of a GABA-ergic agonist.

2. The method of claim 1, wherein the age-related decrease in cortical function comprises a decrease in cognitive function.

3. The method of claim 1, wherein the age-related decrease in cortical function comprises a decrease in sensory function.

4. The method of claim 1, wherein the age-related decrease in visual function comprises a decrease in orientation and direction selectivity.

5. The method of claim 1, wherein the GABA-ergic agonist comprises a GABA a, GABA b, or GABA c receptor agonist.

6. The method of claim 1, wherein the GABA-ergic agonist comprises a benzodiazepine receptor agonist.

7. The method of claim 1, wherein the GABA-ergic agonist comprises GABA, muscimol, baclofen, CaCa, valproic acid, a barbiturate, a benzodiazepine, gabapentin, tigabine, or vigabatrin.

8. The method of claim 1, further comprising determining, prior to treating the subject, whether the subject has an age-related decrease in GABA-ergic activity.

9. The method of claim 1, further comprising determining, prior to treating the subject, whether the subject has an age-related decrease in visual orientation and direction selectivity.

10. A method of treating age related visual decline in a subject, comprising:
determining whether the visual decline comprises an age-related decrease in visual orientation and direction selectivity;
administering a GABA-ergic agonist to the subject in a therapeutically effective amount, sufficient to improve visual orientation and direction selectivity.

11. A method of treating age related cognitive decline in a subject, comprising:
determining whether the cognitive decline comprises an age-related decrease in cognition;
administering a GABA-ergic agonist to the subject in a therapeutically effective amount, sufficient to improve cognition.

12. A method of screening for agents to inhibit age related cortical decline, comprising:
determining whether a test agent increases GABA-ergic cortical inhibitory activity.

13. The method of claim 12, wherein the age related cortical decline comprises an age related decrease in sensory, motor or language function.

14. The method of claim 12, wherein determining whether a test agent increases GABA-ergic cortical inhibitory activity comprises administering the test agent and determining whether the test agent increases a signal to noise ratio.

15. The method of claim 14, wherein determining whether a test agent increases GABA-ergic cortical inhibitory activity comprises measuring a cortical activity with a microelectrode in a neuron.

16. The method of claim 15, wherein the electrode is placed in a neuron having a specific sensory, motor or language function.

17. The method of claim 16, wherein the function is one or more of auditory discrimination of frequency discrimination and/or sound localization, somatosensory function, motor function, or a language area of the cortex.

18. The method of claim 17, wherein the function is assessed by determining a signal to noise ratio in the neuron.

19. The method of claim 17, wherein the function is somatosensory function.

20. The method of claim 19, wherein the somatosensory function is one or more of sensory quality, intensity, position, or stereognosis.

21. The method of claim 17, wherein the function is a language area, and the language area is one or more of Broca's area or Werneke's area.

22. The method of claim 17, wherein the function is motor function, and the function is control of voluntary movements.

23. The method of claim 17, wherein the function is a visual function.

24. The method of claim 23, wherein the visual function is one or more of orientation bias or direction bias.

25. The method of claim 1, wherein the age related decrease in cortical function is one or more of auditory discrimination of frequency discrimination and/or sound localization, somatosensory function, motor function, or a language area of the cortex.

26. The method of claim 25, wherein the function is assessed by determining a signal to noise ratio in the neuron.

27. The method of claim 25, wherein the function is somatosensory function.

28. The method of claim 27, wherein the somatosensory function is one or more of sensory quality, intensity, position, or stereognosis.

29. The method of claim 25, wherein the function is a language area, and the language area is one or more of Broca's area or Werneke's area.

30. The method of claim 25, wherein the function is motor function, and the function is control of voluntary movements.

31. The method of claim 25, wherein the function is a visual function.

32. The method of claim 31, wherein the visual function is one or more of orientation bias or direction bias.

33. The method of claim 1, comprising administering to the subject a therapeutically effective amount of a compound consisting essentially of a GABA-ergic agonist.

34. The method of claim 10, comprising administering a compound consisting essentially of a GABA-ergic agonist to the subject in a therapeutically effective amount.

35. The method of claim 11, comprising administering a compound consisting essentially of a GABA-ergic agonist to the subject in a therapeutically effective amount.
